(19) United States
(12) Patent Application Publication
(10) Pub. No.: US 2024/0277441 A1
(43) Pub. Date: Aug. 22, 2024

(54) ENDOTHERAPY INSTRUMENT HOLDER

(71) Applicant: Keymed (Medical & Industrial Equipment) Limited, Southend-on-Sea (GB)

(72) Inventor: Stuart HOWSDEN, Southend-on-Sea (GB)

(73) Assignee: Keymed (Medical & Industrial Equipment) Limited, Southend-on-Sea (GB)

(21) Appl. No.: 18/437,589

(22) Filed: Feb. 9, 2024

Related U.S. Application Data
(60) Provisional application No. 63/447,390, filed on Feb. 22, 2023.

(30) Foreign Application Priority Data
Feb. 23, 2023 (GB) 2302576.0

(51) Int. Cl. A61B 50/33; A61B 50/26

(52) U.S. Cl. CPC A61B 50/33; A61B 50/26; A61B 2050/318

(57) ABSTRACT

A medical instrument holder including a sheet with front and rear faces and first and second ends. A first pocket is formed on the rear face at the first end and has a mouth which is open towards the second end. A second pocket is formed on the front face at the second end and comprises a mouth which is open towards the first end. An instrument tray is attached to the sheet on the front face at the first end, opposite to the first pocket.

### CROSS REFERENCE TO RELATED APPLICATION

The present application is based upon and claims the benefit of priority from U.S. Provisional Application No. 63/447,390, filed on Feb. 22, 2023, the entire contents of which is incorporated herein by reference.

### BACKGROUND

### Field

The present disclosure relates to an endotherapy instrument holder for use during a medical procedure.

### Prior Art

During an endoscopy procedure a variety of instruments may be used, all of which must be kept sterile and stored safely but readily accessible for use by medical personnel. Such instruments include endoscopes themselves, which have a long flexible insertion tube connected to a handle. The insertion tube may be at least 1-2 m in length. Other elongate flexible tools, such as guide wires and biopsy tools, may also be used which can be passed down through an endoscope for carrying out certain procedures. These may also comprise some form of handle and a flexible elongate member.

Such instruments may be provided in a coiled state and placed in a tray ready for use or uncoiled and hooked over other equipment. However, this presents a number of potential risks. Coiled instruments may uncoil themselves and protrude or fall out of a tray. Instruments which are hooked over other items of equipment may slip off and fall to the ground. Medical personnel may use improvised or unconventional methods to try and hold the instruments in place, such as placing another item on top of a coiled instrument to weigh it down. Nevertheless, there remains a risk that the instrument may uncoil or fall, risking damage to the instrument and contact with other non-sterile items, rendering the instrument unusable. There is also a risk of injury to medical personnel if an instrument suddenly uncoils or falls and they try to catch it or trip over it.

There is therefore a need to provide an effective instrument retention apparatus for aseptic storage and reuse of endotherapy instruments during a medical procedure.

### SUMMARY

The present disclosure provides a medical instrument holder as claimed in claim 1 and medical equipment as claimed in claim 10. Further advantageous features are set out in the dependent claims.

### BRIEF DESCRIPTION OF THE DRAWINGS

The disclosure will now be described in detail, by way of example only, with reference to the accompanying drawings in which:

FIG. 1 is a perspective view of one embodiment of an instrument holder in accordance with the present disclosure;

FIG. 2 shows an embodiment of instrument bag with the instrument holder;

FIG. 3 shows perspective and side views of a folded sheet suitable for forming another embodiment of instrument holder in accordance with the present disclosure;

FIG. 4 is a perspective view of the folded sheet of FIG. 3 with the instrument tray attached;

FIG. 5 is a front view of the folded sheet and instrument tray of FIG. 4 with adjacent edges sealed together to create the pockets;

FIG. 6 shows plan, side and end views of one example of instrument tray;

FIGS. 7a-7b, 8 and 9 show examples of instrument tray attachable to various forms of mounting plate;

FIG. 10 is a perspective view of one form of support for the instrument holder;

FIG. 11 is a perspective of the support of FIG. 10 with the instrument holder fitted;

FIG. 12 is a side view of the support panel of the support of FIG. 10 with the instrument tray in position.

### DETAILED DESCRIPTION

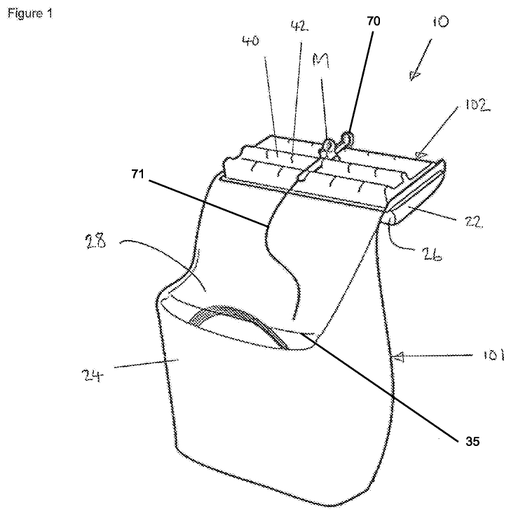

An endotherapy instrument holder 10 in accordance with one embodiment of the present disclosure is shown in FIG. 1. The holder 10 comprises an instrument bag 101 and an instrument tray 102.

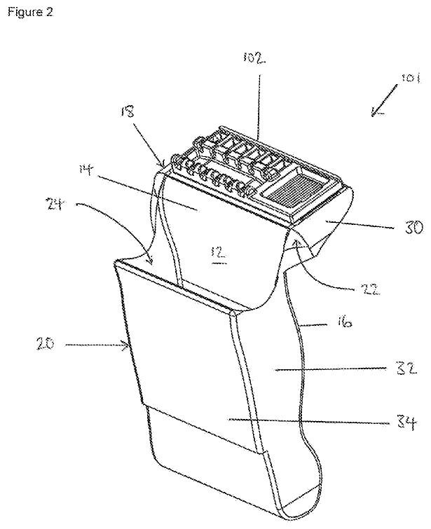

Another embodiment of instrument bag 101, is shown in FIG. 2. The bag 101 may comprise a flexible impermeable sheet 12. The sheet 12 can be formed of a thin plastic film, for example low density polyethylene, although other materials may also be used. The sheet 12 has front and rear faces 14, 16 and first and second ends 18, 20. A first pocket 22 is formed at the first end 18 of the sheet 12 and adjacent the rear face 16. A second pocket 24 is formed at the second end 20 of the sheet 12 adjacent to the front face 14.

The first pocket 22 may be integrally formed with the sheet 12. For example, it may be formed by folding over a first portion 12a of the sheet 12 at the first end 18 towards the rear face 16 and joining the edges of the first portion 12a to the adjacent edges of the sheet 12 to create the first pocket 22. The pocket 22 has an open mouth 26 facing in a direction from the first end 18 towards the second end 20.

Similarly, the second pocket 24 may be integrally formed with the sheet 12 and may be created by folding over a second portion 12b of the sheet 12 at the second end 20 towards the front face 14 and joining the edges together to create the second pocket 24. The second pocket 24 has an open mouth 28 facing in a direction from the second end 20 towards the first end 18.

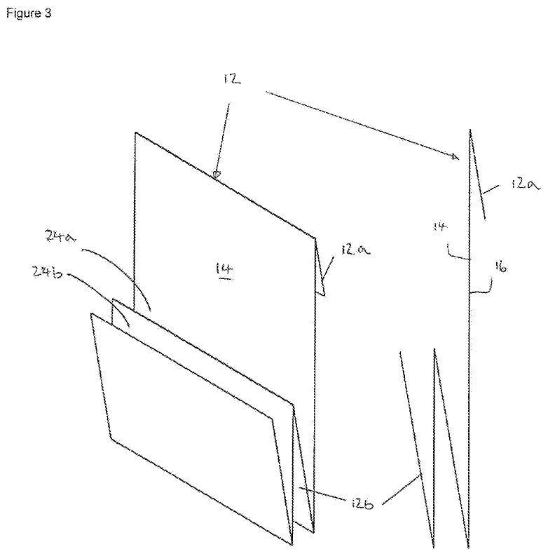

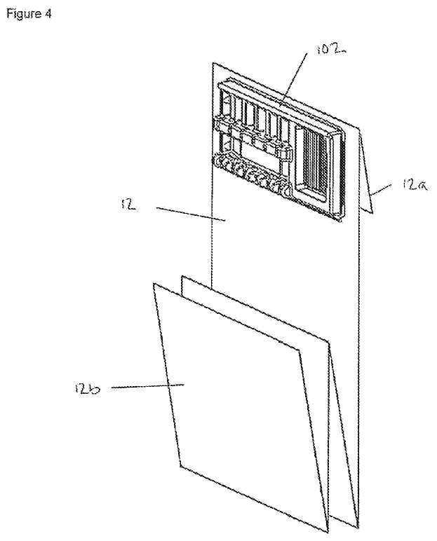

FIG. 3 shows an example of a folded sheet 12 before the edges are joined together to form the pockets 22, 24. In this example, the second portion 12b is folded in a zig-zag form to create two pockets 24a, 24b. Further folds could be provided to create more pockets if required. FIG. 4 shows the folded sheet with the instrument tray 102 attached.

To increase the volume or capacity of each pocket 22, 24 they may be formed with side walls as shown in FIG. 2. For example, the first pocket 22 may have side walls 30 and the second pocket 24 may have side walls 32.

Additional compartments may be formed by attaching one or more additional sheets 34 to form one or more dividing walls 35 within the second pocket 24 as seen in FIG. 1, or on the exterior surface of the second pocket 24, as shown in FIG. 2.

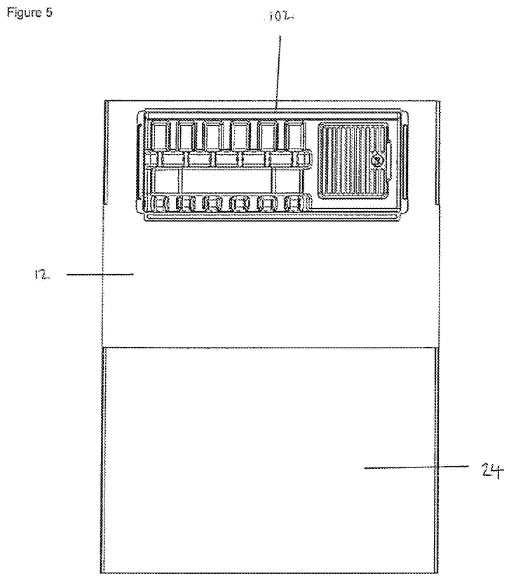

Thus, the sheet 12 and the various pockets 22, 24 may be formed from a single piece of film material which is folded, and the edges sealed together, as mentioned above and as shown in FIG. 5. Alternatively, it may be formed by a number of separate pieces which are joined together at the edges.

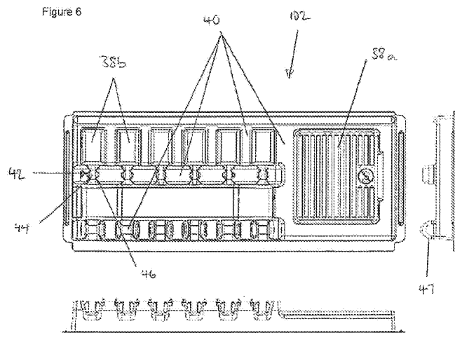

As shown in FIG. 6, the instrument tray 102 is a substantially rigid tray which may be divided into various cavities 38a, 38b separated by walls 40 to accommodate different items. The tray 102 may be moulded and formed of a material such as high-impact polystyrene (HIP). It may be coated with another material, such as polyethylene terephthalate (PET), to facilitate its attachment to the bag 101 by a heat-scaling process.

In the example in FIG. 6, the tray 102 comprises a large cavity 38a which may be used for items such as swabs, and a plurality of smaller cavities 38b to accommodate parts of medical instruments. At least some of the walls 40 may be formed with gripping features 42 for securely holding parts of various instruments, as described below. In other embodiments the large cavity 38a may be omitted and the tray 102 may be formed simply with a number of walls 40 with gripping features 42 which receive parts of medical instruments M, as shown in FIG. 1.

The gripping features 42 may comprise one or more slots 44 with protrusions 46 or textured side walls, dimensioned to grip an instrument pressed between them into the slot 44. Gripping features 42 of different shapes and sizes may be provided to accommodate different instruments. At least some of the walls 40 may have portions 47 formed with a semi-circular or other arcuate profile which is shaped and dimensioned to receive a C-shaped clip attached to a medical instrument.

The tray 102 is attached to the bag 101 at the first end 18 on the front face 14, that is, on the opposite side of the sheet 12 to the first pocket 22. The tray 102 may be adhered or bonded to the bag 101. The tray 102 may be attached to the bag 101 along one of its edges, for example the edge closest to the second pocket 24.

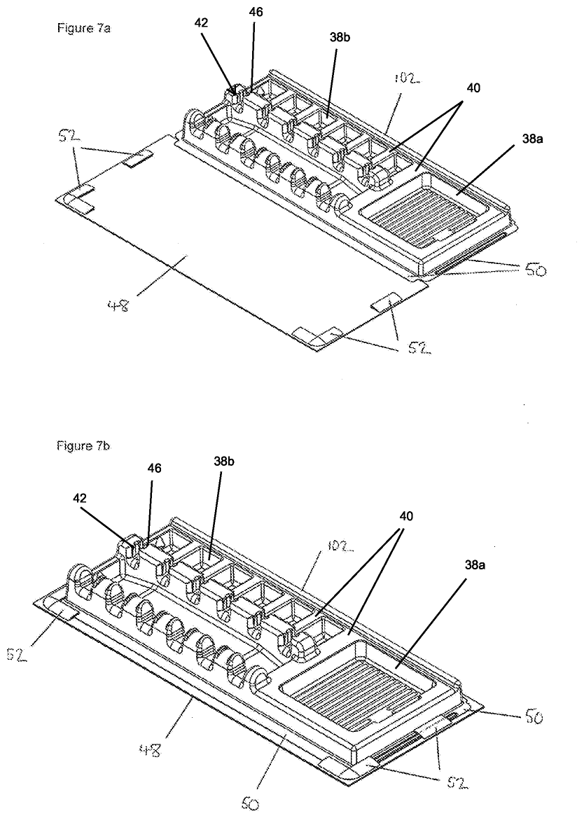

In some embodiments, the tray 102 may be attached to a mounting plate 48 which is itself attached to the bag 101, to provide additional strength and stiffness. For example, as shown in FIGS. 7a and 7b, the tray 102 may be formed with peripheral flanges 50 which slot into louvres 52 formed on the mounting plate 48. In another example, as shown in FIGS. 8, protrusions 54 on the underside of the tray 102 may clip into openings in the mounting plate 48. In another example, shown in FIG. 9, the tray 102 may comprise flanges with projecting ribs 58 which clip into slots 60 on the mounting plate 48.

In some embodiments, the tray 102 may be attached to a mounting plate 48 which is itself attached to the bag 101, to provide additional strength and stiffness. For example, as shown in FIGS. 7a and 7b, the tray 102 may be formed with peripheral flanges 50 which slot into louvres 52 formed on the mounting plate 48. In another example, as shown in FIGS. 8, protrusions 54 on the underside of the tray 102 may clip into openings in the mounting plate 48. In another example, shown in FIG. 9, the tray 102 may comprise flanges with projecting ribs 58 which clip into slots 60 on the mounting plate 48.

In use, the instrument holder 10 may initially be folded up and provided in a sterile package. After removal from the package and unfolding, the instrument holder 10 can be fitted over a support 62. This may be an item of equipment specifically designed for use with the instrument holder 10. Alternatively, the instrument holder 10 may be used with other items of equipment or furniture which serve additional functions, e.g. a workstation intended to hold a number of different pieces of equipment, or a boom arm attached to another structure.

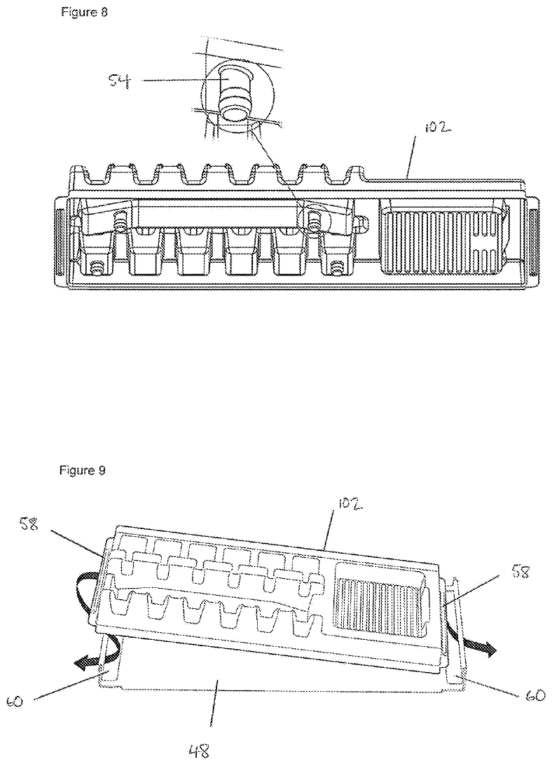

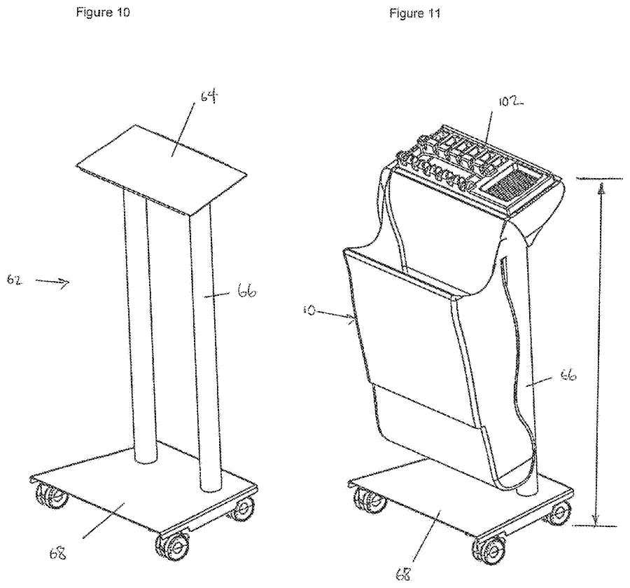

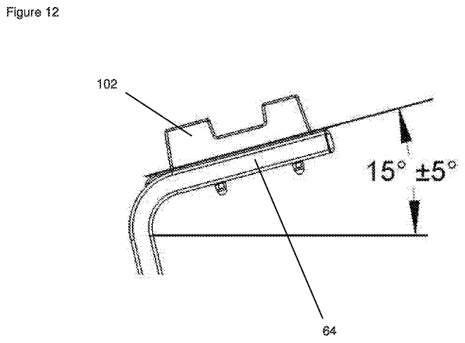

An example support 62 is a trolley or workstation as shown in FIG. 10. This support 62 comprises an upper support panel 64 mounted on one or more legs 66 attached to a base 68, which may have wheels. The upper support panel 64 may be horizontal or tilted at a small angle such as 15±5° to the horizontal for case of use, as shown in FIG. 12. The support 62 may be configured such that, in use, a rear edge of the tray 102 will be positioned between about 895 mm and 995 mm above ground level (this dimension is indicated by the double headed arrow in FIG. 11). For example, the tray 102 may be positioned at about 945 mm above ground level. However, it should be noted that the precise configuration of the support 62 may vary.

The holder 10 is fitted over the workstation 62 by fitting the sheet 12 over the support panel 64 so that the upper support panel 64 is located inside the first pocket 22 and the sheet 12 covers the top surface of the upper support panel 64. The tray 101 will then locate on top of the upper support panel 64. The remainder of the bag 101 hangs downwardly from the upper support panel 64, typically in front of the workstation legs 66, so that the second pocket 24 hangs substantially at the front of the workstation 62 with the open mouth 28 of the second pocket 24 facing upwardly and below the support panel 64 and the tray 102.

Endotherapy instruments 70 can be accommodated in the holder 10 by placing part of the instrument M, such as a rigid handle, in the tray 102, securing it with the gripping features 42, and with the flexible elongate part 71 of the instrument allowed to hang down into the second pocket 24. Different instruments may be positioned in different compartments 24a, 24b etc. of the second pocket 24 to keep them separate from one another. Coiled instruments may also be placed in second pocket 24 without also being attached to the tray 102, as seen in FIG. 1. Thus, the elongate parts of the various instruments do not contact one another, other equipment, or the floor. Items which become dislodged and fall off the tray 102 will fall into the pocket 24, keeping them clean and avoiding contact with the floor. Any fluids on the instruments will drip into the pocket 24 so that they are contained and do not drip on to the floor.

After use, the medical instruments are removed, the holder 10 is removed from the support 62 by unhooking the first pocket 22 from the support panel 64. The holder 10 may be disposed of as clinical waste. However, depending on the materials used, the ability to clean and sterilise the holder may also be provided for.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A medical instrument holder comprising:
a sheet, the sheet having a front face and a rear face, a first end and a second end, wherein a first pocket is formed on the rear face of the sheet at the first end and a second pocket is formed on the front face of the sheet at the second end, the first pocket comprises a mouth which is open towards the second end of the sheet and the second pocket comprises a mouth which is open towards the first end of the sheet, and
a tray attached to the sheet on the front face at the first end, opposite to the first pocket.

2. The medical instrument holder according to claim 1, wherein the tray comprises a plurality of upstanding walls, and at least some walls further comprise a grip configured to hold a medical instrument.

3. The medical instrument holder according to claim 2, wherein the grip comprises slots formed with protrusions configured to grip the medical instrument pressed between the protrusions.

4. The medical instrument holder according to claim 2, wherein the tray further comprises a plurality of cavities between the plurality of upstanding walls.

5. The medical instrument holder according to claim 2, wherein at least some of the plurality of upstanding walls are formed with an arcuate profile configured to receive a C-shaped clip attached to the medical instrument.

6. The medical instrument holder according to claim 1, wherein the second pocket is divided into a plurality of compartments.

7. The medical instrument holder according to claim 1, wherein portions of the sheet are folded over and sealed to a remainder of the sheet to create the first and second pockets.

8. The medical instrument holder according to claim 1, wherein the sheet comprises a flexible impermeable material.

9. The medical instrument holder according to claim 8, further comprising additional pieces of flexible impermeable material attached to the sheet to close sides of one or more of the first and second pockets.

10. A medical equipment comprising:
a workstation; and
the medical instrument holder according to claim 1 mounted on the workstation,
wherein the workstation comprises an upper support plate mounted on a base and the upper support plate is received in the first pocket of the medical instrument holder with the sheet covering the upper support plate and the tray located on the upper support plate, and the second pocket hanging from the upper support plate.

11. The medical equipment according to claim 10, wherein the upper support plate is configured to support the tray at 15±5° to a horizontal.

12. The medical equipment according to claim 10, wherein the upper support plate is configured to support the tray with a rear edge of the tray between about 895 mm and about 995 mm above ground level.
